(19) United States
(12) Patent Application Publication
(10) Pub. No.: US 2017/0360355 A1
(43) Pub. Date: Dec. 21, 2017

(54) METHODS AND SYSTEMS OF SCREENING AND MONITORING ALZHEIMER'S DISEASE USING THE KING-DEVICK TEST

(71) Applicant: King-Devick Test, Inc., Oakbrook Terrace, IL (US)

(72) Inventor: Steven Devick, Oakbrook Terrace, IL (US)

(21) Appl. No.: 15/625,729

(22) Filed: Jun. 16, 2017

Related U.S. Application Data
(60) Provisional application No. 62/351,266, filed on Jun. 16, 2016.

(51) Int. Cl. A61B 5/00; A61B 5/16

(52) U.S. Cl. CPC A61B 5/4088; A61B 5/16

(57) ABSTRACT

A method and system of screening and monitoring a person for Alzheimer's disease, Mild Cognitive Impairment, and Lewy Body Dementia (Dementia) using the King-Devick Test are provided including providing a visual display and a timing device; displaying on the visual display one or more series of symbols arranged in a pattern; providing an indication to the person being screened to begin reading aloud the symbols arranged in the pattern; capturing with the timing device the completion time to read the one or more series of symbols arranged in the pattern; comparing the completion time with a baseline time to complete reading the one or more series of symbols; and determining whether the person has an “abnormal” result based on comparing the completion time with the baseline time.

### CROSS-REFERENCE TO RELATED APPLICATIONS

This application claims priority to U.S. Provisional Patent Application No. 62/351,266 filed on Jun. 16, 2016, entitled “ALZHEIMER'S DISEASE SCREENING USING THE KING-DEVICK TEST,” which is hereby incorporated by reference in its entirety herein.

### TECHNICAL FIELD

The present disclosure relates generally to techniques for screening and monitoring Dementia such as Alzheimer's Disease, Mild Cognitive Impairment, Lewy Body Dementia and other dementia.

### BACKGROUND

Alzheimer's Disease (AD) affects an estimated 5.5 million Americans, of which 5.3 million are 65 and older. Given the overall aging of the population, the number of new cases of AD is expected to increase significantly. Total healthcare payments in 2017 for all individuals with AD, Mild Cognitive Impairment (MCI), Lewy Body Dementia (LBD) or other dementias (collectively referred to herein as “Dementia”) is estimated to exceed $259 billion. In general, health care costs increase with the presence of Dementia. For example, people with Dementia have twice as many hospital stays per year as other older people. Moreover, people with Dementia make up a large proportion of all elderly people who receive adult day services and nursing home care.

Although there is currently no cure for AD, a number of therapies are being developed to help with both cognitive and behavioral symptoms. However, such treatments are effective to temporarily slow the worsening of symptoms when administered in the early stages of the disease. It is now believed that AD pathology may be present up to 20 years before clinical symptoms are observed. Consequently, AD remains underdiagnosed in nearly half of the American population where currently available treatments may be helpful.

There is a need for sensitive and readily available screening tools that can detect AD in its early stages (e.g., MCI). Advances in techniques for the early detection and diagnosis of AD are proceeding. Biological markers are currently being studied for their ability to indicate early stages of AD. For example, currently available techniques, such as neuroimaging, including magnetic resonance imaging (MM), computer tomography (CT) and positron emission tomography (PET), and lumbar puncture cerebrospinal fluid protein (CSF) analysis, are invasive, expensive, and time consuming.

The use of neuropsychological tests and related paradigms are noninvasive, inexpensive, and sensitive to early AD-related cognitive changes. However, neuropsychological testing requires in-depth training to ensure standardized administration and accurate interpretation of findings, and is labor intensive. As such, neuropsychological testing is not typically feasible for use in a generalized clinical setting, such as primary care. Primary care is indeed a central location for the diagnosis and management of Dementia, with annual wellness visits now requiring the addition of cognitive evaluation.

A brief, noninvasive test that is rapid and easy to administer, and is sensitive to detection of Dementia is needed.

### SUMMARY

A method of screening and monitoring a person for Dementia using the King-Devick Test (“K-D Test” or the “Test”) is provided including providing a visual display and a timing device; displaying on the visual display one or more series of symbols arranged in a pattern; providing an indication to the person being screened for Dementia to read aloud the symbols arranged in the pattern; capturing with the timing device the completion time to read the one or more series of symbols arranged in the pattern; comparing the completion time with a baseline time to complete reading the one or more series of symbols; and determining whether the person has an “abnormal” Dementia result based on comparing the completion time with the baseline time.

In some embodiments, the symbols are randomly- or pseudo-randomly generated numbers. The symbols can be colors or pictures. In some embodiment, three series of symbols are displayed sequentially, wherein the series of symbols is arranged in order of increasing difficulty.

In some embodiments, the baseline time corresponds to prior completion times for the person being screened for Dementia. In some embodiments, the baseline time corresponds to normative values.

In some embodiments, the method further includes providing a computer, wherein the visual display is a computer screen. The timer device can be a timer on the computer. In some embodiments, the visual display is a printed card. In some embodiments, the timing device is a stopwatch.

In another aspect, the method further includes recording the number of errors by the person while reading the series of symbols arranged in the pattern, comparing the number or errors with a baseline number of errors made while reading the series of symbols arranged in the pattern; and determining whether the person has an “abnormal” Dementia result based on comparing the number or errors with a baseline number of errors made. In some embodiments, the baseline number of errors corresponds to prior number of errors made by the person being screened for Dementia. In some embodiments, the baseline number of errors corresponds to normative values.

In a further aspect, the method includes administering a treatment for Dementia to the person if the result is “abnormal.” In some embodiments, the method further includes recommending the person undergo additional testing for Dementia before administering a treatment for Dementia to the person if the result is “abnormal.” The treatment can be administered to the person substantially immediately after an “abnormal” result is obtained. The treatment can be administered to the person before any other signs or symptoms of Dementia are observed. The method can be administered to the person at regular intervals to detect minute changes in the person's Dementia status and/or response to therapy.

A method of screening and monitoring a person for Dementia using the King-Devick Test is provided. The method is implemented on a computer system having one or more physical processors configured by machine-readable instructions which, when executed, perform the method of providing a visual display and a timing device; displaying on the visual display the one or more series of symbols arranged in a pattern; providing an indication to the person being screened for Dementia to begin reading aloud the symbols arranged in the pattern; capturing with the timing device the completion time to read the one or more series of symbols arranged in the pattern; comparing the completion time with a baseline time to complete reading the one or more series of symbols; and determining whether the person has an “abnormal” Dementia result based on comparing the completion time and errors performance with the baseline performance.

### BRIEF DESCRIPTION OF THE DRAWINGS

The foregoing and other objects, features and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein.

FIGS. 1-4 depict an exemplary arrangement of symbols as displayed in a K-D Test.

FIG. 5 depicts a system for screening patients in accordance with exemplary embodiments of the disclosed subject matter.

FIG. 6 depicts exemplary instructions for a person in accordance with exemplary embodiments of the disclosed subject matter.

### DETAILED DESCRIPTION

The embodiments will now be described more fully hereinafter with reference to the accompanying figures, in which exemplary embodiments are shown. The foregoing may, however, be embodied in many different forms and should not be construed as limited to the illustrated embodiments set forth herein.

The K-D was developed in the early 1980's as an objective tool to evaluate saccadic eye movements. The K-D Test is widely used by reading teachers in schools, medical professionals, or parents at home to determine whether a student's poor reading performance is related to deficiencies in their ability to move their eyes efficiently. Children that score below the expected norm are, almost always, not efficient readers.

The inventor first realized that his K-D Test, widely recognized as a tool to evaluate saccadic eye movements, is ideally suited for use as a rapid, easy-to-administer screening tool to evaluate individuals having, suspected of having, or at risk for developing Dementia. Under normal conditions, the K-D Test is administered, and an objective conclusion is reached, in around one minute for adults. The K-D Test can therefore be used in a variety of situations in which rapid, easy to administer AD screening is beneficial, such as during an examination by a medical professional or as part of a home- or self-examination program designed for people having, suspected of having, or at risk for developing AD, MCI or other forms of Dementia like LBD.

In one embodiment, the K-D Test is administered as part of general screening examinations to healthy patient populations in order to provide a rapid, objective, and easy-to-administer screen for Dementia or other causes of suboptimal nervous system function.

In another embodiment, the K-D Test is administered to persons having Dementia, suspected of having Dementia, or at risk for developing Dementia as a method for early detection of Dementia, or as a method to monitor progression of Dementia and/or quality of life. Because oculomotor dysfunctions are a common early biomarker of AD and other forms of Dementia, the K-D Test is particularly useful as an objective method to screen for Dementia. In this scenario, the K-D Test is administered soon after the patient or a person observing the patient first notices any subtle Dementia symptoms, and the result informs the patient whether or not he or she should seek medical treatment for Dementia. Moreover, due to its high level of sensitivity, the K-D Test is also administered to patients at risk for developing Dementia (e.g., middle-aged or geriatric patients, patients with genetic predisposition for AD, and patients with a family history of AD) before the onset of any perceivable Dementia symptoms, regardless of how subtle, providing a very early method of detection. Such at-risk persons could self-administer the K-D Test (or have it administered to them) every day, regardless of whether they feel well, as an easy way to detect minute changes in Dementia status. Those results could be electronically transmitted to the person's doctor if there is a reduction in performance time or errors in order to help the doctor make treatment decisions.

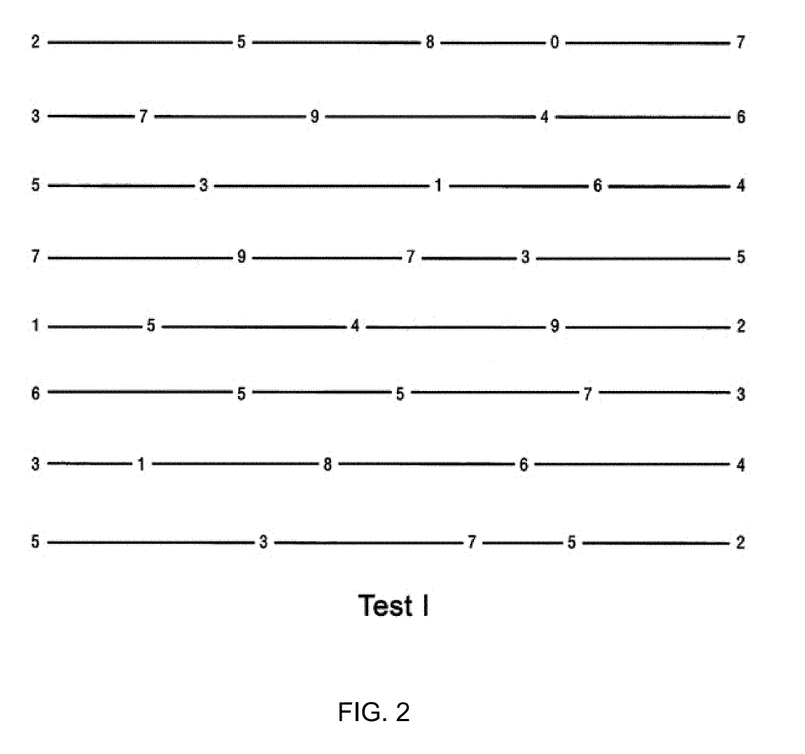

The K-D Test is administered by providing a display and a timing device. In some embodiments, a system 100 is provided, as illustrated in FIG. 2, which includes a tablet 110 or other computer device having one or more physical processors configured by software which, when executed, perform the operations described herein. The tablet 110 has a user interface 120 including a touch-sensitive display and a timer (not shown, but integrated into the microprocessor of the tablet 110). The display 120 provides a series of symbols arranged to require the person to use saccadic eye movements in order to read the symbols quickly and accurately. In some embodiments, the symbols are random numbers, letters, colors, or pictures. Reading aloud is understood to mean verbally identifying the number or letter and/or naming the color or picture image, e.g., “One,” “Red,” or “Apple.”

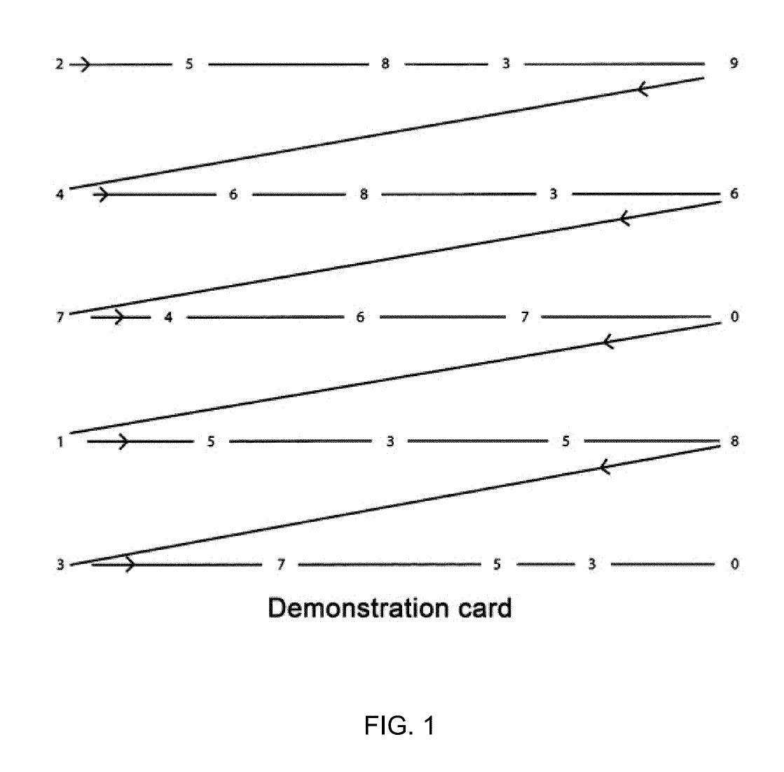

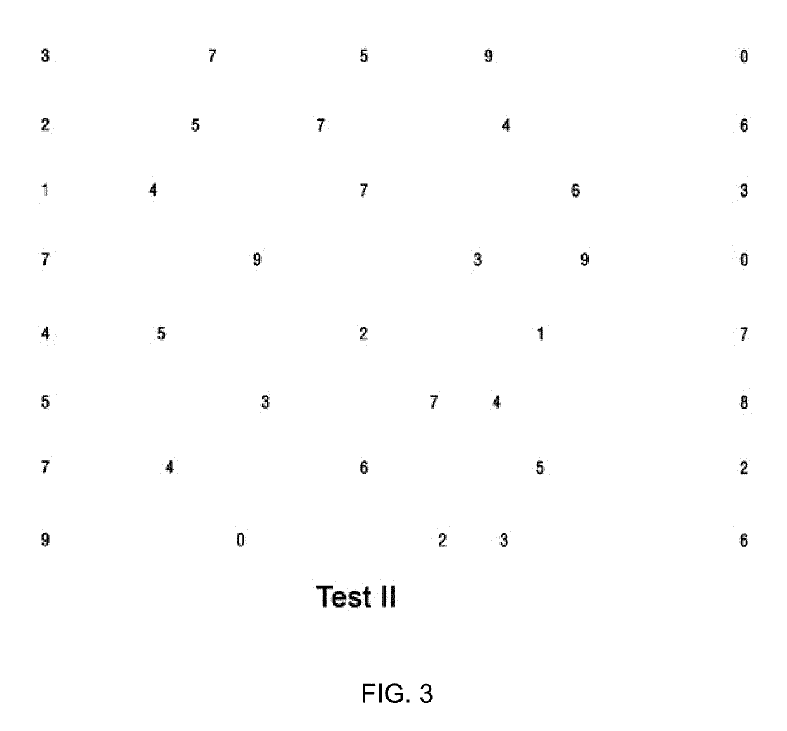

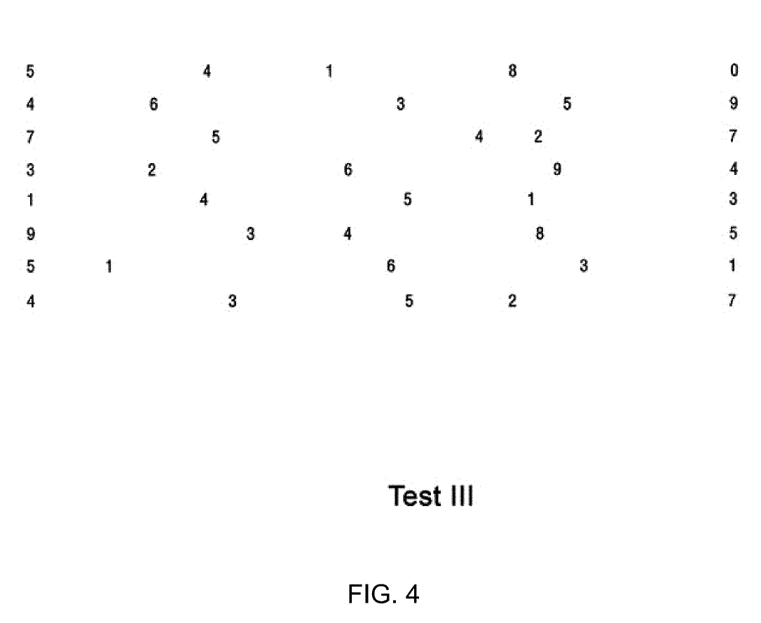

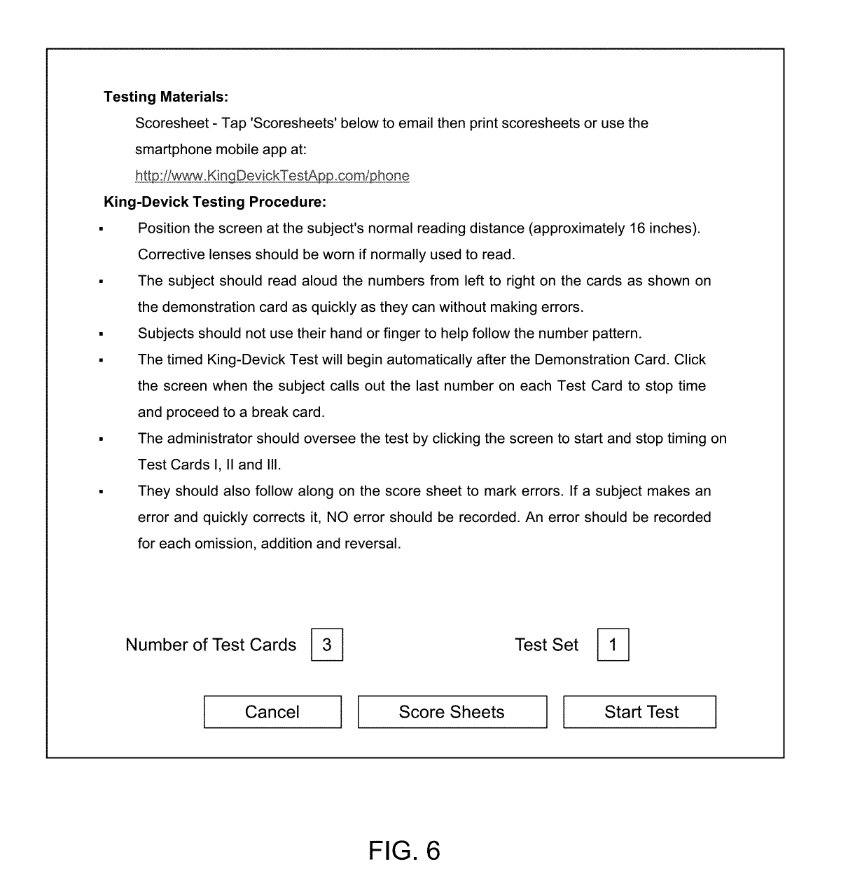

A demonstration arrangement of symbols is shown in FIG. 1, which includes a series of numbers arranged in rows, with lead-lines to assist the person to read the arrangement of number in the pattern shown, i.e., in rows from top to bottom. During the Test, the person receives instructions to read aloud symbols provided on the display in a specified order. The person can be directed not to move their head during the Test. Exemplary instructions are illustrated in FIG. 6, which may be displayed prior to the commencement of the Test. The timer is used measure the amount of time needed by the person to complete the Test. In some embodiments, the K-D Test uses three test screens display sequentially on the tablet 110. Each test screen is progressively harder to read than the previous. For example, the first screen has horizontal lines that connect the symbols in rows (FIG. 2). The second test screen omits the lines connecting the symbols (FIG. 3), and the third test screen is hardest yet, because the rows of symbols are closer together which creates visual crowding (FIG. 4). During the Test, the person is instructed to read several arrangements of symbols (also referred to interchangeably as “test cards” or “test screens”), e.g., Test screen I (FIG. 2), followed by Test screen II (FIG. 3), and followed by Test screen III (FIG. 4). For certain test subjects, only a single test screen is used, e.g., for subjects with more advanced Dementia.

The time to complete the Test is compared to baseline data. The timer begins recording time once the first arrangement of symbols or test screen is displayed. In some embodiments, the touch-sensitive display is used by the person to advance from one test screen to the next test screen, and then to stop elapsed time when the final test screen is completed. The timer can be paused between test screens. For example, the time is paused when “break” screens are displayed between successive test screens. The total time necessary for the person to complete all test screens is recorded as the completion time. In some embodiments, the Test is administered on a series of printed cards. In some embodiments, the timing device is a stopwatch.

A Test administrator can use on the timer to time the Test and follow along on an answer key. The answer key lists the correct sequence of symbols for each Test. The number of errors made during the Test is also recorded by a test administrator.

The test data is compared to baseline data. A result is considered “normal” if the completion time is the same or shorter than the baseline time and the total number of errors is below the baseline number of errors. A result is considered “abnormal” if the total time is longer than the baseline time and/or the total number of errors is greater than the baseline number of errors. A slight decline in performance as a person ages is normal.

The baseline data can include normative data, e.g., a statistical data for the person's age group, regarding the range of time necessary to complete the Test and the number of errors that a person of an age group can score and still have a “normal” Test result. Alternatively, the baseline data may be for a particular person's performance on the Test. In this case, baseline measurements may be made for the person, during, for example, routine medical examinations or at some other time. The individual's baseline data would include the total time to take the Test and the number of errors made while taking the Test. If multiple Tests were taken, the baseline time and number of errors could be averages. If there is an extended time period between tests, the baseline results may be adjusted to account for increasing age of the person being tested. Those baseline results would then be made available by way of the person's medical records or other method of recordation for purposes of comparison if needed.

A determination about whether the completion time and number of errors is “normal” or “abnormal” is made by comparison to a chart. Such chart may be uploaded onto the tablet or computer, and may include either the individualized baseline data, or generalized baseline data for the person's demographic, e.g., age. In some embodiments, the processor is able provide a determination about whether a completion time is “normal” or “abnormal” by a comparison the completion time to the baseline time.

K-D Test results for a person testing “abnormal” would then serve as information that is submitted to the person's physician and such person may be treated as if the person has Dementia, or is at risk for developing Dementia. Such treatment could include recommending the person undergo additional testing for Dementia, referring the person to a neurologist or other specialist for additional testing for Dementia, recommending the person be administered a treatment for Dementia, or administering the person a treatment for Dementia, etc. The K-D Test can be administered to the person periodically, e.g., daily or weekly, to detect minute changes in the person's Dementia status.

For patients who take the Test regularly, or frequently enough that they might learn the number patterns and thereby misleadingly improve their scores (perhaps even as their saccadic eye movements degrade), a battery of K-D Tests can be used, in which the spacing of the symbols is preserved but the symbols themselves are changed randomly or pseudo-randomly. Administering the Test on computer, such as on a suitably-sized tablet computer, can facilitate the use of a virtually limitless number variations.

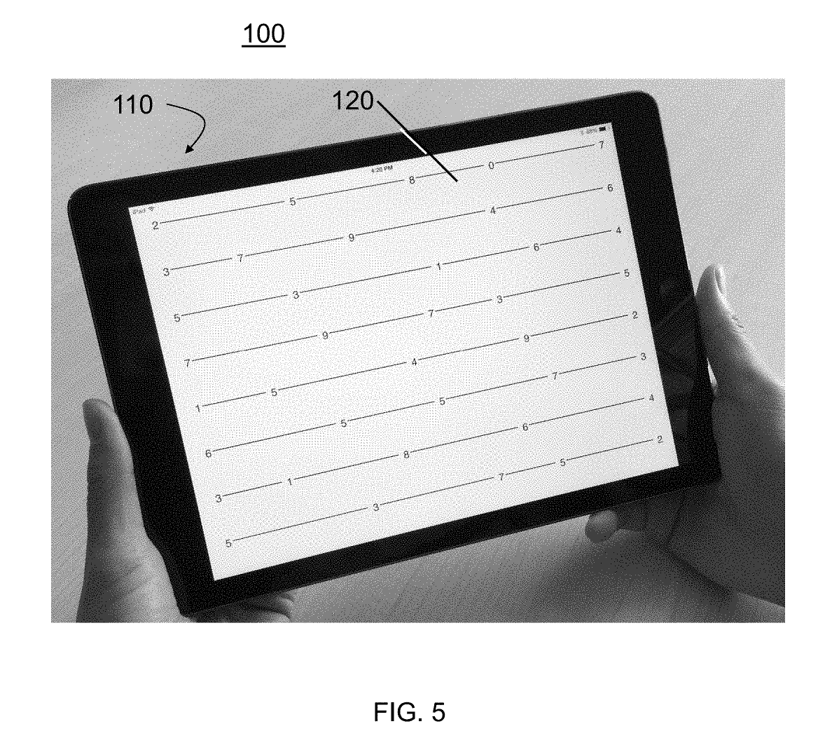

System 100 as illustrated in FIG. 5, screens and monitors a person for Dementia using the King-Devick Test. System 100 includes a computer system, such as a tablet 110 having a visual display and a timing device and one or more physical processors configured by machine-readable instructions to display on the visual display the one or more test screens. The system 100 further provides instructions, as illustrated in FIG. 6, as an indication to the person being screened to begin reading aloud the symbols arranged in the pattern. A timing device captures the completion time to read the one or more series of symbols arranged in the pattern. In some embodiments, the system 100 includes a database with which to compare the completion time with a baseline time to complete reading the one or more series of symbols. In some embodiments, the system 100 can make a determination whether the person has an “abnormal” result based on comparing the completion time with the baseline time. For, example if the time for the person to complete the Test exceeds the baseline time.

The above systems, devices, methods, processes, and the like may be realized in hardware, software, or any combination of these suitable for an application. The hardware may include a general-purpose computer and/or dedicated computing device. This includes realization in one or more microprocessors, microcontrollers, embedded microcontrollers, programmable digital signal processors or other programmable devices or processing circuitry, along with internal and/or external memory. It will further be appreciated that a realization of the processes or devices described above may include computer-executable code created using a structured programming language that may be stored, compiled or interpreted to run on one of the above devices, as well as heterogeneous combinations of processors, processor architectures, or combinations of different hardware and software. In another aspect, the methods may be embodied in systems that perform the steps thereof, and may be distributed across devices in several ways. At the same time, processing may be distributed across devices such as the various systems described above, or all the functionality may be integrated into a dedicated, standalone device or other hardware. In another aspect, means for performing the steps associated with the processes described above may include any of the hardware and/or software described above. All such permutations and combinations are intended to fall within the scope of the present disclosure.

Embodiments disclosed herein may include computer program products comprising computer-executable code or computer-usable code that, when executing on one or more computing devices, performs any and/or all the steps thereof. The code may be stored in a non-transitory fashion in a computer memory, which may be a memory from which the program executes (such as random access memory associated with a processor), or a storage device such as a disk drive, flash memory or any other optical, electromagnetic, magnetic, infrared or other device or combination of devices. In another aspect, any of the systems and methods described above may be embodied in any suitable transmission or propagation medium carrying computer-executable code and/or any inputs or outputs from same.

It will be appreciated that the devices, systems, and methods described above are set forth by way of example and not of limitation. Absent an explicit indication to the contrary, the disclosed steps may be modified, supplemented, omitted, and/or re-ordered without departing from the scope of this disclosure. Numerous variations, additions, omissions, and other modifications will be apparent to one of ordinary skill in the art. In addition, the order or presentation of method steps in the description and drawings above is not intended to require this order of performing the recited steps unless an order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the disclosure as defined by the following claims, which are to be interpreted in the broadest sense allowable by law.

What is claimed is:

1. A method of screening and monitoring a person for Alzheimer's disease, Mild Cognitive Impairment, and Lewy Body Dementia (Dementia) using the King-Devick Test, comprising:
providing a visual display and a timing device;
displaying on the visual display one or more series of symbols arranged in a pattern;
providing an indication to the person to begin reading aloud the symbols arranged in the pattern;
capturing with the timing device the completion time to read the one or more series of symbols arranged in the pattern;
comparing the completion time with a baseline time to complete reading the one or more series of symbols; and
determining whether the person has an “abnormal” result based on comparing the completion time with the baseline time.

2. The method of claim 1, wherein the symbols are randomly- or pseudo-randomly generated numbers.

3. The method of claim 1, wherein the symbols are colors or pictures.

4. The method of claim 1, wherein displaying on the visual display one or more series of symbols arranged in a pattern comprises displaying three series of symbols arranged in order of increasing difficulty.

5. The method of claim 1, wherein the baseline time corresponds to prior completion times for the person.

6. The method of claim 5, wherein the baseline times is adjusted for the age of the person.

7. The method of claim 1, wherein the baseline time corresponds to normative values.

8. The method of claim 1, further comprising providing a computer,
wherein the visual display is a computer screen.

9. The method of claim 8, wherein the timing device is a timer on the computer.

10. The method of claim 1, wherein the visual display is a printed card.

11. The method of claim 1, wherein the timing device is a stopwatch.

12. The method of claim 1, further comprising
recording the number of errors by the person while reading the series of symbols arranged in the pattern;
comparing the number or errors with a baseline number of errors made while reading the series of symbols arranged in the pattern; and
determining whether the person has an “abnormal” result based on comparing the number or errors with a baseline number of errors made.

13. The method of claim 12, wherein the baseline number of errors corresponds to prior number of errors made the person.

14. The method of claim 12, wherein the baseline number of errors corresponds to normative values.

15. The method of claim 1, further comprising
administering a treatment to the person if the result is “abnormal.”

16. The method of claim 15, wherein administering a treatment comprises recommending the person undergo additional testing before administering a treatment to the person if the result is “abnormal.”

17. The method of claim 15, wherein the administering a treatment occurs substantially immediately after an “abnormal” result is obtained.

18. The method of claim 15, wherein the administering a treatment occurs before any other signs or symptoms of Dementia are observed.

19. The method of claim 12, comprising repeating the method of claim 12 periodically to observe changes in completion time and number of errors made over time.

20. A method of screening and monitoring a person for Alzheimer's disease, Mild Cognitive Impairment, and Lewy Body Dementia (Dementia) using the King-Devick Test, the method being implemented on a computer system having one or more physical processors configured by machine-readable instructions which, when executed, perform the method, further comprising:
providing a visual display and a timing device;
displaying on the visual display one or more series of symbols arranged in a pattern;
providing an indication to the person being screened to begin reading aloud the symbols arranged in the pattern;
capturing with the timing device the completion time to read the one or more series of symbols arranged in the pattern;
comparing the completion time with a baseline time to complete reading the one or more series of symbols; and
determining whether the person has an “abnormal” result based on comparing the completion time with the baseline time.

21. The method of claim 20, wherein the symbols are randomly- or pseudo-randomly generated numbers.

22. The method of claim 20, wherein the symbols are colors or pictures.

23. The method of claim 20, wherein the baseline time corresponds to prior completion times for the person being screened.

24. The method of claim 23, wherein the baseline time is adjusted for the age of the person.

25. The method of claim 20, wherein the baseline time corresponds to normative values.

26. The method of claim 20, further comprising
recording the number of errors by the person while reading the series of symbols arranged in the pattern;
comparing the number or errors with a baseline number of errors made while reading the series of symbols arranged in the pattern; and
determining whether the person has an “abnormal” result based on comparing the number or errors with a baseline number of errors made.

27. A system of screening and monitoring a person for Alzheimer's disease, Mild Cognitive Impairment, and Lewy Body Dementia (Dementia) using the King-Devick Test, comprising
a visual display;
a timing device; and
a computer system having one or more physical processors configured by machine-readable instructions to:
display on the visual display one or more series of symbols arranged in a pattern.
provide an indication to the person being screened to begin reading aloud the symbols arranged in the pattern;
capture with the timing device the completion time to read the one or more series of symbols arranged in the pattern;
compare the completion time with a baseline time to complete reading the one or more series of symbols; and
determine whether the person has an “abnormal” result based on comparing the completion time with the baseline time.
